(19) United States
(12) Patent Application Publication
(10) Pub. No.: US 2013/0041036 A1
(43) Pub. Date: Feb. 14, 2013

(54) FERULIC ACID AS FEED SUPPLEMENT IN BEEF CATTLE TO PROMOTE ANIMAL GROWTH AND IMPROVE THE MEAT QUALITY OF THE CARCASS AND THE MEAT

(71) Applicants: Humberto González Rios, Morelos (MX); Diego Arturo Gil Lozano, Morelos (MX); Antonino Berrondo Mir, Morelos (MX)

(72) Inventors: Humberto González Rios, Morelos (MX); Diego Arturo Gil Lozano, Morelos (MX); Antonino Berrondo Mir, Morelos (MX)

(73) Assignee: BIOKAB, S.A. DE C.V., Mexico City (MX)

(21) Appl. No.: 13/446,594

(22) Filed: Apr. 13, 2012

(30) Foreign Application Priority Data
Aug. 10, 2011 (MX) MX/A/2011/008722

(51) Int. Cl. A61K 31/192; A61P 21/06

(52) U.S. Cl. 514/570

(57) ABSTRACT

The present disclosure comprises a natural method of feed supplement to beef cattle, with a maize extract obtained from the nixtamalization process with a high content of ferulic acid and its salts, for a period of 30 days in the final phase of the feedlot. Use of this dietary supplementation enables an increase in carcass yield and meat quality that is not negatively affected as with a commercial β-agonist. In addition to the above effects, the effective supplementation of ferulic acid to cattle is shown to have antioxidant effects on fresh meat during its shelf life, which can lead to great benefits for the industrial meat market.

### CROSS-REFERENCE TO RELATED APPLICATIONS

Not Applicable.

### STATEMENT REGARDING FEDERALLY SPONSORED RESEARCH OR DEVELOPMENT

Not Applicable.

### FIELD OF THE INVENTION

The present disclosure corresponds to the field of feed supplements used to improve feed efficiency, promote animal growth and change the meat quality of animals under intensive feeding systems.

### BACKGROUND

In the last few years, several countries of global importance in beef production have implemented strategies based on reproductive, nutritional, environmental and pharmacological techniques that enhance the assimilation of food.

Some compounds such as antibiotics, probiotics, and anabolic agents improve the metabolism resulting in higher feed efficiency, and with this increased weight gain in shorter periods with large economic benefits. However, meat quality is a consumer demand that beef producers must consider, since the use of hormonal compounds and/or food additives in animals can affect physical, chemical and sensory characteristics of the meat (Eng, K. 2000. Choices of implants, implants strategies Increases again. Feedstuffs. 72:10).

One of the growth promoters currently used by producers of beef cattle are known as adrenergic β-agonists, which are analogues to catecholamine, epinephrine and norepinephrine hormones. The β-agonists improve nitrogen retention and reduce fat deposition in animals (Mersmann, H. J. 1998, Beta-Adrenergic Receptor Modulation of Adipocyte Metabolism Modulation and Growth, Journal Animal Science. 80: E. Suppl. 1, E24-E29). Due to the efficiency in the productive performance, in countries like Mexico and the United States the use of zilpaterol hydrochloride and ractopamine hydrochloride compounds is authorized in cattle (Norma Oficial Mexicana-NOM-EM-015-ZOO-2002. Especificaciones técnicas para el control y use de beta-agonistas en los animales. SAGARPA. México, D.F).

Despite the remarkable benefits on productive efficiency of the authorized β-agonists, there is a rejection by the meat consumers of the consumption of meat from animals treated with these compounds, which is due to the recent reports of intoxications with meat or viscera, caused by misuse by the producers (Sumano, L. H.; C. L. Ocampo y O. L. Gutiérrez. 2002. Clembuterol y otros β-agonistas, [symbol] una opción para la producción pecuaria o un riesgo para la salud pública? Veterinaria México. Vol. 33, Num. 2). In the particular case of Mexico, in the last few years there have been cases of human poisoning, due to the consumption of meat or viscera of animals that have been fattened with β-agonist clembuterol, which is not allowed for use in animal production, however, some beef cattle producers have been using it irresponsibly and without any technical restrictions. The consumption of meat from animals treated with high amounts of clembuterol may result in severe effects on the consumer, such as thyroid gland alterations, metabolic disorders or temperature intolerance. Excessive levels of this drug may further cause irregular heart rate, nervousness, involuntary shaking of hands or feet, headache, increased sweating, insomnia, potential muscle spasms, increased blood pressure and nausea. Due to these problems, most of the regulatory agencies in different countries have forbidden its use in animal feeding. Likewise, countries of the European Union and some Asian countries prohibit the use of β-agonist compounds ractopamine, salbutamol, apart from clembuterol.

In general, the quality and chemical composition of the meat is affected by several factors, such as feeding, sex, age, animal breed, muscle:fat ratio, muscle location, as well as the use of growth promoters

Among the attributes that influence consumer acceptance, tenderness, juiciness and the taste of the cooked meat stand out. Of these three factors tenderness plays the most decisive role (Kemp, C. M., P. L. Sensky, R. G. Bardsley, P. J. Buttery y Tim Parr. 2010. Tenderness—An enzymatic view. Meat Science. 84:248-256). Another characteristic of meat quality, and that is valued by the consumer at the time of purchase, is the color of the meat, which is considered one of their preferred criteria.

On the other hand, research reports (Avendaño-Reyes, L., V. Torres-Rodriguez, F. J. Meraz-Murillo, C. Pérez-Linares, F. Figueroa-Saavedra, and P. H. Robinson. 2006. Effects of two β-adrenergic agonists on finishing performance, carcass characteristics, and meat quality of feedlot steers. Journal of Animal Science. 84:3259-3265) have indicated that some characteristics of meat quality can be affected by the use of β-agonist compounds, showing a significant increase in the shear force of meat from steers supplemented with ractopamine compared to the control.

Due to this problem meat producers must find new alternatives in which natural anabolic compounds are used, with no impact on consumer health, and that furthermore, the main characteristics of quality are not negatively affected.

A naturally occurring compound with a chemical structure analogue to β-agonists is ferulic acid, present in fruits, cereals, and grains and seeds being the richest source. This compound is the active component of gamma oryzanol, commonly used as a dietary supplement by athletes to increase muscle mass. Ferulic acid structure as shown in Illustration 1 contains a phenolic ring, which is why it is mentioned in some research that it is a bioactive compound due to its antioxidant capacity and that its addition to foods inhibits lipid peroxide formation.

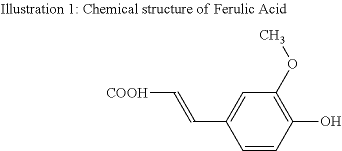

Currently ferulic acid is commercialized in nutritional supplements for humans with the aim of increasing lean muscle growth, attributed to its anabolic effect (Yagi, K., and Ohishi, N. 1979. Action of ferulic acid and its derivatives as antioxidants. The Journal of Nutritional Science Vitaminology, 25, 127-130). In the patent of Henry Classen and Hongyu Qiao (Sinapic acid supplementation, US20080113003) it is proposed the use of a supplement containing ferulic acid plus sinapic acid, to be used in monogastric animals in order to promote a more favorable microbial ecology in the digestive tract of the animals, and said patent does not indicate that the supplement has functions as a growth promoter.

Another Patent report (Liu Yaguang, 4945115), indicates the use of a pharmaceutical composition in tablet form containing ferulic acid, used to decrease the effects of anticancer chemotherapy and improve the immune function in humans.

The use of ferulic acid in animal production is virtually nil. The patent developed by Herrera, H., Alejo M. L., & Asaff, A. J. (AA61K31192F1, Jan. 24, 2011. Methods to accelerate muscle development decrease fat deposits, and enhance feeding efficiency in pigs) reports that when supplementing with 50 ppm of ferulic acid there is a decrease in the thickness of the dorsal fat of pigs, however, it does not indicate that the supplement can be used for the same purpose in beef cattle.

There is a report of an approved Japanese patent (JP-H06-153 815 A. Method for improving meat quality), which uses a feed supplement for Japanese cattle (the Wagyu breed) in the final stage of intensive feed lot and shows an improvement in the red color of the meat and maintaining thereof during the product's shelf life. However, that patent differs from the present disclosure in several important elements. The Japanese patent indicates the use of a food additive the last 30 days of intensive feedlot, and said additive is composed of two antioxidants, ferulic acid and E vitamin. In the case of the present disclosure, the supplement is composed solely of ferulic acid and its salts.

They reported (JP-H06-153 815 A) that the supplement is helpful for improving the red color of the meat and maintaining it during the shelf life, without reporting any growth promoting effect or improving the quality of the carcass, since it was not used for that purpose, as opposed to the innovative part of this proposal which showed the effect of promoting growth and improvement in the quality of beef carcasses and some sensory attributes of the meat, which are listed in the claims.

With respect to the dose of the supplement used in animals, they reported a dose of ferulic acid from 0.5 to 10 mg/kg body weight of the animal/day, equivalent to a dose between 225 to 4,500 mg/animal/day, for animals of approximately 450 kg in the final phase of fattening; and this dosage is complemented or reinforced with a daily supplement of 675 to 4,500 mg/animal/day of vitamin E. However, our proposed method suggests the use of a maximum of 3,500 mg/animal/day of ferulic acid, which is obviously lower than the combined dose of the two antioxidant of the Japanese patented method, and this may be attributable to the degree of purity of the active compound, because they do not indicate the purity of the compounds, while the proposed supplement molecule has a purity exceeding 95%.

Additionally, the evidence of the example reported here indicates that there are improvements in carcass yield and sensory characteristics of the meat (tenderness and taste), which had not been previously claimed.

The ferulic acid has been noted for its ability to decrease substances reactive to oxygen, fulfilling the same function as superoxide dismutase, the enzymes that protect living beings of the substances reactive to oxygen.

Ferulic acid and its salts has the ability to donate protons, be a good oxidant and thus prevents the reactions of free radicals. For these properties, the ferulic acid is listed in food additives as an “oxidation inhibitor” that can be used as an antioxidant or anti-bleach with many patents.

Ferulic acid has also been pointed out as an agent that: 1) protects the liver from toxic compounds, 2) protects the muscular system from wear, 3) prevents colds and flu due to its antimicrobial properties, 4) has anti-inflammatory properties and 5) has ergogenic properties to promote muscle development (WO 2008/116319 A1).

To extract ferulic acid from natural sources hydrolytic methods are used, either the alkaline or enzymatic type. After obtaining the hydrolysates, is proceeded to its recovery and purification. Until now the properties of cinnamic acid derivatives were studied based on the pure compounds. However, mixtures of active ingredients contained in alkaline or enzymatic plant extracts, such as those contained in the nejayote and concentrates, allow us to reach effective doses that are related to the size and weight of the animal, which had not been studied.

In the example of the disclosure, pure or substantially pure ferulic acid is used as a feed supplement in beef cattle under intensive feeding conditions, to promote animal growth and/or improve some quality characteristics of the carcass or the meat. In the study where embodiments of the invention were tested, strict scientific procedures were followed in order to assure the validity of the information.

### BRIEF SUMMARY

In particular the disclosure is related to the use of plant extracts with structural characteristics similar to synthetic compounds identified as β-agonists and used as animal growth promoters in intensive meat production systems.

The invented formulation is used as a feed supplement for beef cattle, and is composed of alkaline extracts obtained from maize grains during the nixtamalization process, which contains concentrates of ferulic acid crystals of high purity obtained from the same nejayote and other active compounds of interest.

This natural extract rich in ferulic acid, used as a feed supplement in doses of 250 mg/kg of food, during the final phase of the feedlot (last 30 days) of commercial beef cattle, shows growth promoting activity similar to commercial β-agonists compounds without compromising the quality characteristics of the meat such as sensory tenderness, shear force texture, and also shows to have antioxidant activity, reducing oxidative deterioration and maintaining the fresh color of the meat during its shelf life.

An embodiment includes providing a natural feed supplement containing ferulic acid at a dose of 250 mg/kg feed, during the last phase of feedlot of commercial beef cattle, in order to achieve a carcass yield similar to that obtained with commercial β-agonists compounds, with the advantage that ferulic acid is natural and does not affect meat tenderness as commercial products do. Additionally, the ferulic acid supplementation shows a significant effect as antioxidant, since it maintained with fewer changes the characteristics of color and lipid oxidation of meat during storage, helping to extend the shelf life of the meat. The above being a great competitive advantage for cattle producers and fresh meat traders.

One of the objectives of the disclosure is to provide a natural animal supplement containing a concentration of at least one cinnamic acid derivative, such as the trans-ferulic acid for use as a dietary supplement in beef cattle in order to improve their productive performance.

Under these considerations, the disclosure additionally provides a method that promotes the growth of beef cattle, by administering a dietary supplement containing an effective amount of ferulic acid.

In particular, the present disclosure allows the significant increase in carcass yield (carcass weight/live weight ratio) of beef cattle under intensive fattening in feedlot, through the effective ferulic acid supplementation to the commercial cattle.

Another objective is to provide a natural method to improve meat quality through the administration of a feed supplement with ferulic acid to the beef cattle, which produces meat with improved quality characteristics in terms of sensory tenderness and instrumental texture.

Another of the objectives is to provide a method (with a natural non-hormonal component) of animal food supplementation that benefits the shelf life of fresh beef from animals produced under these conditions during its cold storage.

### BRIEF DESCRIPTION OF DRAWINGS

A complete understanding of the optimized design and fabrication methods of the present disclosure and its various features, object and advantages may be obtained from the illustrations of the following drawings:

FIG. 1 illustrates the performance of the color a* parameter, which measures the tendency to redness (Y-axis) over cold storage time (X-axis).

FIG. 2 illustrates the performance of the variable TBA (Y-axis) of fresh meat, which is indicative of the oxidative deterioration of meat over cold storage time (X-axis).

FIG. 3 illustrates a photographic pattern of changes of meat color (Y-axis) during the study of shelf life for each of the experimental treatments (X axis).

### NOTATION AND NOMENCLATURE

Certain terms are used throughout the following descriptions and claims to refer to particular system components. This document does not intend to distinguish between components that differ in name but not function.

The term “effective amount” refers to the amount of compound that is enough to obtain the intended beneficial effect. In this context, it is considered that a sufficient beneficial effect is present if one or more of the effects detailed above are achieved. In particular it is considered that a beneficial effect is present if the treatment offers a financial return at least equal to the cost of the treatment, preferably at least three times the cost of the treatment. To those skilled in the art, the effective amount will depend on the species of animal, duration of the treatment and other factors.

The term “pure ferulic acid” refers to the trans-ferulic acid or any salts thereof, with a purity higher than 95% obtained from natural sources or chemical synthesis. Trans-ferulic acid is also known as trans-4-hydroxy-3-methoxy cinnamic acid, and is a crystalline solid with a melting point of 170° C. (Beilstein Index: 10,436; Merck Index (14): 4062). This compound derived from cinnamic acid is widely distributed in nature being part of the cell wall of many plant species, without any reported adverse side effects when consumed by animals or humans. Instead, numerous studies have shown that ferulic acid has several beneficial properties on both human and animal health, and is considered a nutraceutic (Fazzary and Ju; Acta Biochimica et Biophysica Sinica 2007, 39: 811-828).

The term “extract” refers to the product from the enzymatic or alkaline hydrolysis of plant material, containing free trans-ferulic acid and that has been treated to remove some of the liquid medium in which this compound is dissolved by any unitary operation known, such as evaporation in any modality, reverse osmosis, etc.; or extracted from hydrolyzed plant material by a solvent like alcohol or ethyl acetate, by any known methods of extraction.

### DETAILED DESCRIPTION

The present disclosure comprises a method for administering to animals, particularly beef cattle (commercial breeds) during the last phase of intensive fattening, a dietary supplement containing an “effective amount” of pure ferulic acid at 100 to 250 mg/kg of food. In addition to the multiple properties that have been identified, the present disclosure shows the ability of ferulic acid to act as a growth promoter in animals, particularly beef cattle. By having a high homology in their hydrocarbon chain with drugs such as ractopamine hydrochloride and zilpaterol hydrochloride, ferulic acid probably also acts as an agonist of β-adrenergic receptors, but unlike synthetic drugs, it is a natural product without the described side effects.

The compositions of the present disclosure are looking for a beneficial effect on the quality of the carcass and meat of beef cattle fed under intensive conditions in feedlot.

The use of natural compounds with chemical structures similar to that of β-agonists of adrenergic receptors may fulfill the function of increasing carcass yields, without the negative effects of, for example, clembuterol (toxic) or other compounds such as zilpaterol hydrochloride and ractopamine hydrochloride which increase the toughness of the meat.

Those skilled in the art will notice that the disclosure described herein is susceptible to variations and other modifications from those specifically described. The disclosure includes such variations and modifications. The disclosure also includes all the steps, information, formulations and compounds.

The present disclosure involves feed compositions including a mixture of feed materials with ferulic acid and its salts or formulations of these in a suitable carrier. Ferulic acid is preferably administered to cattle receiving a diet rich in protein and energy in order to promote muscle development.

Accordingly, a part of the disclosure is to provide a food preparation, to which ferulic acid and its salts or formulations thereof have been added in a carrier or vehicle suitable to administer the proper dosage to the animals in question.

The amount of ferulic acid and its salts added to the feed preparation should be enough to achieve concentrations between 100 and 250 mg, preferably between 150 and 200 mg of ferulic acid in said food preparation.

The uniform incorporation of a feed supplement containing ferulic acid in the integral ration of animals, promotes a uniform distribution of the active ingredient in the final food with which they are mixed. Therefore, the carriers play an important role to ensure adequate distribution of the active ingredient in all the food.

The feed for beef cattle supplemented with ferulic acid, in its preferred embodiment generally contains between 100 and 250 mg of active compound per kilogram of food, preferably expressed as: 150 to 200 g per ton of feed.

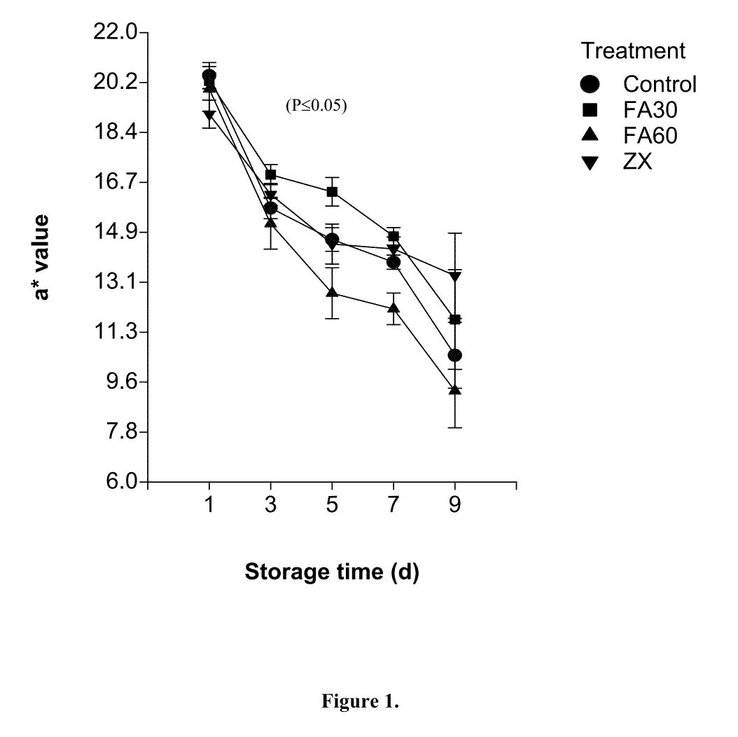

The effects of incorporation of a feed supplement containing ferulic acid may be found for example in FIGS. 1-3. More specifically, FIG. 1 shows the performance of the color a* parameter, which measures the tendency to redness (Y axis) over cold storage time, which is indicated on the X axis. If the values of a* are decreased dramatically during storage, it is an indication that the meat has lost its cherry red color it had at the beginning of the storage, so the decline of this parameter during cold storage should be avoided as much as possible. The meat with the ferulic acid treatment for a 30 day period during fattening (FA30), shows to have a delayed effect of the deterioration of the red color of the meat.

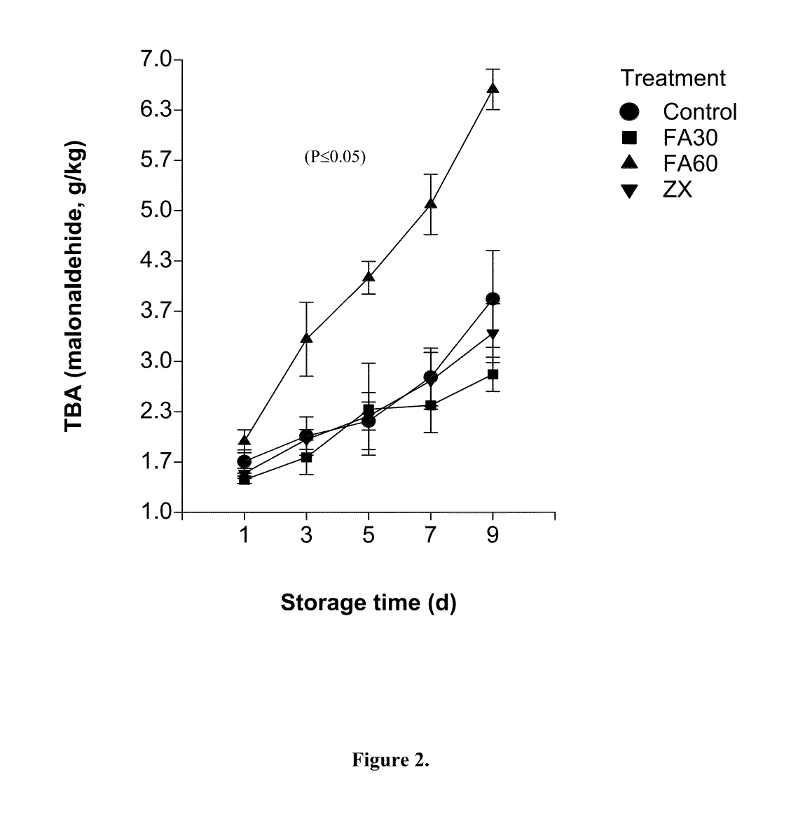

Additionally, FIG. 2 presents the performance of the variable TBA (Y axis) of fresh meat, which is indicative of the oxidative deterioration of meat over cold storage time (X axis). If TBA values remain lower, it is an indication that the meat has oxidized less and therefore remains in better conditions for its commercialization. TBA values during shelf life are lower for meat from animals supplemented with ferulic acid for 30 days in the final stage of fattening.

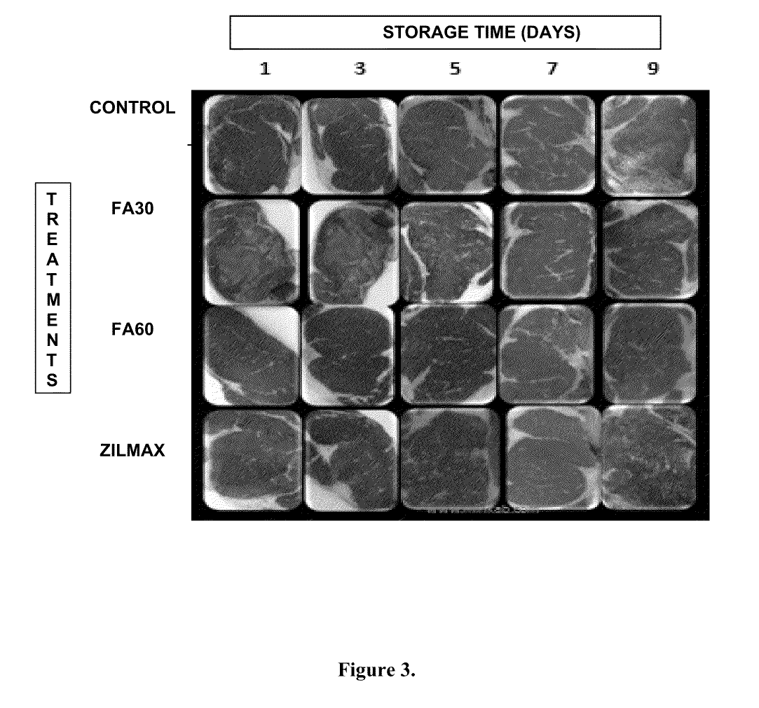

Finally, FIG. 3 shows a photographic pattern of changes of meat color during the study of shelf life for each of the experimental treatments. The columns (Y axis) indicate the color of the meat for each of the treatments on a particular day of the refrigerated storage. The rows or lines (X axis) show color changes overtime of storage for each of the experimental treatments. In this photographic pattern, it is observed that meat from animals supplemented with 30 days of ferulic acid (FA30), maintain the red meat color with less changes until day 7 of storage.

While the preferred embodiments of the disclosure have been shown and described, modifications thereof can be made by one skilled in the art without departing from the spirit and teachings of the disclosure. The embodiments described and the examples provided herein are exemplary only, and are not intended to be limiting. Many variations and modifications of the disclosure herein are possible and are within the scope of the disclosure. Accordingly, the scope of protection is not limited by the description set out above, but is only limited by the claims which follow, that scope including all equivalents of the subject matter of the claims. To further illustrate various illustrative embodiments of the present disclosure, the following examples are provided.

### EXAMPLES

In order to demonstrate these effects, that the ferulic acid supplementation has a promoting effect on animal growth and meat quality of cattle, an study was done which used one hundred beef cattle of commercial breeds with an average live weight at the beginning of the test of 450 kg, and with mainly racial influences of European breeds, which were fed under intensive conditions of feedlot production, with a diet high in concentrate. In this study, all animals received the same prophylactic management, and all were subsequently assigned randomly to one of the following four treatments (25 animals per treatment, divided into pens of 5 experimental units per pen):

### Definition of the 4 Treatments

Treatment 1 (Control): control animals (receiving only the basal diet without supplementation of the additive).

Treatment 2 (Ferulic Acid for 30 days=FA30): Animals receiving the basal diet supplemented with 240 ppm of ferulic acid during the last 30 days of the fattening period.

Treatment 3 (Ferulic Acid for 60 days=FA60): Animals receiving the basal diet supplemented with 240 ppm of ferulic acid during the last 60 days of the fattening period.

Treatment 4 (Zilmax commercial Zilpaterp; =ZX): Animals receiving the basal diet and supplemented with 6 ppm hydrochloride zilpaterol during the last 30 days of the fattening period.

During the experimental phase, all animals received a feeding ration consisting of 20% fodder and 80% concentrate. The crop was corn stover, while the concentrate consisted of different proportions of rolled corn grain, soybean meal, canola meal, distillery grains, and molasses. The ration was provided twice a day, and there was free access to the food and drinking water.

Initial and final live weight was recorded in the productive performance trial. The performance test lasted 60 days. Daily feed intake was evaluated, weight was recorded at the beginning and end of experimental period, in order to estimate the average daily gain and feed conversion.

Before concluding the performance test, 2 animals were randomly selected per pen (10 per treatment) for humanitarian slaughter. The animals were sacrificed following the standard procedures of the official Mexican standards in the municipal slaughterhouse of Guadalajara, Jalisco. Animal live weight was recorded before slaughter.

During the slaughter process, the pH of the carcass at 45 min postmortem in the Longissimus dorsi muscle (LD) was measured and the weight of the hot carcass was recorded. The carcasses were refrigerated at 0° C. for 24 hr. After this, the final pH was measured and cold carcass weight recorded, and the classification of carcasses was carried out, recording the Rib eye area (REA) in square inches, back fat thickness (BFT) in mm, and the marbling in the 12th intercostal space of the LD; the skeletal maturity and conformation of the carcass was also evaluated (10 carcasses per treatment). The assessment of quality of the carcass was made following the procedures described by the US Department of Agriculture (USDA, 2000).

Subsequent to the carcass classification the rib eye cut (LD muscle) was obtained of the left side of each selected carcass. Meat samples were identified, vacuum packed, frozen at −18° C., and then shipped under refrigeration by air courier to the Centro de Investigación en Alimentacion y Desarrollo A.C. (CIAD) facilities at Hermosillo, Sonora, to perform quality analysis of the meat: Chemical analysis (moisture and fat content), physicochemical (objective color parameters L*, which measures the brightness of the meat, a* value, which measures the redness, and b* value which measures the tendency towards yellowness, and hue-angle, Warner-Bratzler shear force, water retention capacity and pH). A sensory test was done by a trained panel consisting of 10 members using a descriptive test with a semi-structured 10 cm scale, where zero indicates a demerit of the attribute and a 10 indicates a favorable rating (e.g. tenderness, zero indicates extremely hard and 10 extremely tender). The sensory attributes that were measured: total color and overall appearance of raw meat, flavor, odor, color, tenderness, fat perception, juiciness, and perception of connective tissue in cooked meat. The methodology recommended by AMSA (American Meat Science Association, 1995, Chicago, Ill., EU) was followed for cooking, presentation and evaluation of samples. To measure cooking loss, the meat sample was weighed before and immediately after reaching the final cooking temperature, which was expressed in percentage.

In order to estimate the antioxidant effect of ferulic acid, 5 chops of the meat samples were taken (5 experimental units) of the respective treatments, which were packed with traditional packaging (film wrapped) and subjected to a refrigeration process from 4 and 5° C. for 10 days in a room equipped for this purpose. During the shelf life (Day 1, 3, 5, 7 and 9 of storage) the objective color parameters mentioned above, as well as determination of substances reactive to thiobarbituric acid (TBARS) were evaluated according to the methodology described by Pfalzgraf et al. (1995). TBA values were expressed as substances reactive to 2-thiobarbituric acid (TBARS) in mg malonaldehyde/kg sample.

All data for response variables of the quality of carcass and meat were analyzed under a completely randomized design, with a one-way ANOVA, taking the experimental treatments as a fixed effect. For carcass BFT and REA characteristics the hot carcass weight was used as a covariable. Regarding the sensory attributes, the model also included the effect of panelist as the repeated measure. The variables of the shelf life study were analyzed as a completely randomized design with factorial arrangement, factor A being the treatment with 4 levels, and factor B, the storage time with 5 levels. When there were statistical differences (P<0.05) between treatments, average comparisons were performed by the multiple range test of Tukey. All data was processed in the NCSS statistical package (NCSS, 2001).

Results of the example test are shown in FIGS. 1 to 3 and Tables 1 to 3.

Table 1 shows the results for the quality characteristics of the carcass, which were obtained in the experiment mentioned in the example of the application of the disclosure. The variable carcass yield percentage (CY), which estimates the total percentage of meat for sale that is obtained in relation to its weight before slaughter, was affected by treatments, observing that treatment with ferulic acid during the last 30 days of fattening (FA30) produces carcass yields similar to those obtained from animals supplemented with commercial β-agonist (ZX).

TABLE 1
Quality carcass characteristics by experimental treatment.
TREATMENTS

| Variable* | CONTROL | FA30 | FA60 | ZX | SEM | P value |
|---|---|---|---|---|---|---|
| IW, kg | 448.58 | 452.4 | 448.4 | 448.0 | 14.52 | |
| FW, kg | 547.3 | 553.5 | 551.9 | 579.4 | 16.31 | NS |
| HCW, kg | 320.84 | 336.42 | 330.42 | 356.34 | 9.95 | NS |
| CCW, kg | 315.34 | 331.13 | 325.42 | 350.62 | 9.80 | NS |
| Dressing, % | 1.71 | 1.56 | 1.51 | 1.60 | 0.05 | NS |
| CY, % | 58.65^{a} | 60.77^{b} | 59.85^{ab} | 61.53^{b} | 0.50 | 0.001 |
| PH45 | 6.56^{b} | 6.54^{b} | 6.33^{ab} | 6.17^{a} | 0.08 | 0.004 |
| PH24 | 5.63 | 5.63 | 5.61 | 5.63 | 0.02 | NS |
| REA (Inch²) | 13.67 | 14.54 | 14.21 | 15.41 | 0.56 | NS |
| BFT (mm) | 4.22 | 4.29 | 4.38 | 3.05 | 0.50 | NS |

IW: Initial weight,
FW: final weight,
HCW: hot carcass weight,
CCW: cold carcass weight,
CY: carcass yield,
PH45: pH at 45 min postmortem,
PH24: pH at 24 h postmortem,
REA: rib eye area,
BFT: back fat thickness.
^{ab}Means within rows with different superscript differ (P ≦ 0.05).
SEM: Standard error of mean.

Table 2 shows the results for chemical and physical-chemical quality of the Longissimus dorsi muscle for each treatment tested in the experiment where the disclosure was applied. It is observed that the variable of shear force (SF), which measures the hardness of the meat, presents values significantly lower in meat from animals supplemented with ferulic acid during the last 30 days of fattening, compared to meat animals supplemented with commercial β-agonist (ZX). It is also noted that the color of fresh meat (L*, a*, b* and hue) were not affected by treatments, and the values are within normal ranges for this matter.

TABLE 2
Chemical and Physiochemical quality of Longissimus dorsi muscle by
experimental treatment.
TREATMENTS

| Variable* | CONTROL | FA30 | FA60 | ZX | SEM | P value |
|---|---|---|---|---|---|---|
| Moisture, % | 73.33 | 73.10 | 73.41 | 73.02 | 0.37 | NS |
| Fat | 2.15 | 2.13 | 2.52 | 2.36 | 0.35 | NS |
| content, % | | | | | | |
| pH | 5.60 | 5.54 | 5.57 | 5.57 | 0.06 | NS |
| L* value | 35.86 | 36.66 | 35.74 | 35.34 | 1.07 | NS |
| a* value | 17.29 | 16.86 | 17.55 | 16.39 | 0.80 | NS |
| b* value | 12.69 | 12.53 | 13.22 | 11.95 | 0.58 | NS |
| Hue angle | 36.43 | 36.79 | 37.13 | 36.05 | 1.44 | NS |
| SF, kg F | 9.05^{ab} | 7.94^{a} | 8.32^{ab} | 10.37^{b} | 0.62 | 0.04 |
| Cooking | 16.25^{a} | 17.65^{ab} | 19.65^{b} | 19.20^{b} | 0.71 | 0.01 |
| loss, % | | | | | | |
| WHC, % | 24.37 | 25.73 | 25.44 | 25.13 | 1.57 | NS |

SF: shear force,
WHC: water holding capacity.
^{ab}Means within rows with different superscript differ (P ≦ 0.05).
SEM: Standard error of mean.

Regarding the sensory quality of meat (Table 3), supplementation of ferulic acid and its salts for a period of 30 days in the final stage, resulted in a more tender, juicier and better flavored meat than the meat from animals supplemented with Zilmax. This last, it is an excellent competitive advantage over the commercial product. Both the instrumental and sensory evaluation indicated that the meat of the FA30 treatment, was more tender than that obtained from animals supplemented with Zilmax.

In the study of the shelf life of the Longissimus dorsi muscle, significant changes were observed in the color of the meat and lipid oxidation due to the treatments. These results are shown in FIGS. 1 and 2.

During the shelf life of the meat, color parameter a* (redness) was significantly affected by treatments, observing that meat from animals supplemented with ferulic acid during the last 30 days of fattening (FA30) maintained the highest values of a* until day 7 of storage. The last is favorable, since the color is the main attribute of quality that the consumer takes into account as a purchase decision. FIG. 1.

TABLE 3
Sensorial characteristics of Longissimus dorsi muscle by experimental
treatments.
TREATMENTS

| Variable | CONTROL | FA30 | FA60 | ZX | SEM | P value |
|---|---|---|---|---|---|---|
| Overall Color | 7.68 | 7.51 | 7.65 | 7.41 | 0.15 | NS |
| Appearance | 7.43 | 7.41 | 7.11 | 7.17 | 0.15 | NS |
| Odor intensity | 8.05 | 8.05 | 7.90 | 7.89 | 0.14 | NS |
| Tenderness | 7.01^{ab} | 7.68^{b} | 7.54^{ab} | 6.85^{a} | 0.19 | 0.007 |
| Juiciness | 6.49^{ab} | 7.09^{b} | 6.76^{ab} | 6.00^{a} | 0.21 | 0.003 |
| Flavor intensity | 7.36^{ab} | 7.73^{b} | 7.32^{ab} | 6.56^{a} | 0.21 | 0.002 |
| Fat perception | 3.66 | 3.22 | 3.62 | 3.34 | 0.35 | NS |
| Connective tissue | 1.67 | 1.57 | 1.50 | 1.42 | 0.26 | NS |
| perception | | | | | | |

^{ab}Means within rows with different superscript differ (P ≦ 0.05).

During its shelf life, the meat from animals supplemented with ferulic acid the last 30 days of fattening in feedlot, produced smaller increases in TBA values (FIG. 2), indicating that there was less lipid oxidation, which can be considered as a antioxidant effect, which is beneficial for maintaining the red color of the meat and its fresh smell during its commercialization. By contrast, meat from animals supplemented for 60 days showed a faster oxidation, indicating that a prolonged ferulic acid supplementation may have pro-oxidative effects.

We claim:

1. A method for improving meat quality in beef cattle, which comprises administering a feed supplement comprising ferulic acid or a salt thereof in an amount from 100 to 250 mg.

2. The method according to claim 1, wherein said feed supplement comprises ferulic acid or a salt thereof in an amount from 150 to 200 mg.

3. The method according to claim 1, wherein said feed supplement is administered during the last phase of feedlot.

4. The method according to claim 3, wherein said feed supplement is administered for a period of 20 to 35 days.

5. The method according to claim 1, wherein said feed supplement increases carcass yield and improves the quality of the meat in terms of sensory tenderness and instrumental texture.

6. A food supplement for beef cattle comprising ferulic acid or a salt thereof in an amount from 100 to 250 mg.

7. The food supplement for beef cattle of claim 6, comprising ferulic acid or a salt thereof in an amount from 150 to 200 mg.
